(19) United States
(12) Patent Application Publication
(10) Pub. No.: US 2017/0014543 A1
(43) Pub. Date: Jan. 19, 2017

(54) HYDROGEL BASED ON γ-POLYGLUTAMIC ACID AND ε-POLYLYSINE CROSSLINKED POLYMER, AND PREPARATION METHOD THEREFOR

(71) Applicant: NANJING UNIVERSITY OF TECHNOLOGY, Nanjing, Jiangsu (CN)

(72) Inventors: Hong Xu, Nanjing (CN); Bo Chi, Nanjing (CN); Jun Xia, Nanjing (CN); Sha Li, Nanjing (CN); Xiaohai Feng, Nanjing (CN)

(73) Assignee: NANJING TECH UNIVERSITY, Nanjing (CN)

(21) Appl. No.: 15/100,210

(22) PCT Filed: Dec. 28, 2013
(86) PCT No.: PCT/CN2013/090813

§ 371 Date: May 27, 2016

(30) Foreign Application Priority Data
Dec. 11, 2013 (CN) 201310676374.0

(51) Int. Cl. A61L 15/26; C08J 3/075; C08J 3/24; A61L 15/60

(52) U.S. Cl. CPC A61L 15/26; A61L 15/60; C08J 3/075; C08J 3/246; C08J 2377/04; C08J 2477/04

(57) ABSTRACT

A hydrogel based on a cross-linked γ-polyglutamic acid and ε-polylysine polymer is obtained by cross-linking of γ-polyglutamic acid with ε-polylysine, and it is a polymer having the following constitutional unit, wherein, m is a natural number of 15 to 45, n is a natural number of 3900 to 17000, and x is a natural number of 5 to 40. It also discloses a preparation method of as-described hydrogel and its application in preparation as a medical wound dressing.

This application is the U.S. national phase of International Application No. PCT/CN2013/090813 Filed on 28 Dec. 2013 which designated the U.S. and claims priority to Chinese Application Nos. CN201310676374.0 filed on 11 Dec. 2013, the entire contents of each of which are hereby incorporated by reference.

### TECHNICAL FIELD

The present invention relates to a preparation method of a biocompatible hydrogel, specifically it relates to a preparation method of a hydrogel of a biocompatible γ-polyglutamic acid and ε-polylysine cross-linked polymer obtained by a chemical cross-linking.

### BACKGROUND OF THE INVENTION

Skin is an important organ of human body. For reasons such as wound, burn etc, the skin may get a large range of damage, and may have complications and endanger human life, how to effectively accelerate healing of wound is always a tireless exploration task in the field of medicine. It is considered by the traditional view that, it should create a dry environment for the wound as far as possible, so as to reduce infection and contribute to wound healing. But with the deepening of research, people find that a wet and non-infection environment is most favorable to continuous tissue repair process [Wound repair and regeneration, 2009 (17): 505-510]. Under the guidance of theory of such “wet wound healing”, research and application of a medical polymeric hydrogel wound dressing gradually rises, and gradually replaces the traditional dressing for application in various ulceration and wound.

Hydrogel is a three-dimensional cross-linking network material which is able to absorb and retain a large amount of water while being insoluble in water, its network consists of macromolecular main chain and hydrophilic functional groups, it is a kind of functional polymeric material having both water absorption and water retention, and it has unique properties such as high mechanical strength, biodegradability, high swelling rate, biocompatibility and stimulative responsibility, etc. Polymeric hydrogel wound dressing is one main application of the hydrogel in medical material, it is a kind of new dressing product developed in recent years. Traditionally, a doctor generally treat a wound with a sterile gauze and topical antibiotics, the gauze tend to adhere to skin wound tissue, and tend to destroy new epithelium and granulation tissue at time of dressing change, causing hemorrhage, which not only delays wound healing, but also induces pain to the patient. Hydrogel dressing can effectively overcome above-described shortcomings, it has a good steam and gas permeability, it may not adhere to the wound, and easy to be replaced. Drug component and growth factor can be incorporated into the hydrogel dressing, prompting wound healing, and the heat capacity of hydrogel material per se is large, it has a mild cooling effect on wound, and it may relieve wound pain. Thus, scholars at home and abroad have conducted extensive research on the hydrogel dressing and made great achievements.

γ-polyglutamic acid and ε-polylysine are only two kinds of naturally existing amino-acid homopolymers in 20 kinds of amino acids forming human body so far, both of them are prepared by microbial fermentation. Molecular weight of γ-polyglutamic acid is between 100 thousand to 2 million Daltons, it can be degraded into glutamic acid monomer in vivo, and absorbed by human body, without toxicity and side effects. There are a large number of free carboxyl groups on its molecular chain, being easy to be modified. Molecular weight of the ε-polylysine is between 3000 and 5000 Daltons, it is similar to nature extracellular matrix (ECM) in terms of protein composition and function, its molecular chain has many amino groups, it can effectively promote cell adhesion and growth, furthermore, ε-polylysine per se is a polycation, being easy to have strong electrostatic interaction with polyanion, it has a good penetrability to biomembrane. γ-polyglutamic acid and ε-polylysine have obvious advantages over traditional gel material, both have good in vivo degradability and biocompatibility, they will not induce rejection of human body.

U.S Patent 2003/0211129A1 discloses that preparing a membrane by using polyglutamic acid and polylysine as raw material, making polyglutamic acid and polylysine self-assembled by electrostatic adsorption mechanism, meanwhile adding nanoparticles of ZrO₂, Al₂O₃ and TiO₂ making the raw material self-assembling to a membrane, the membrane may be used as a drug carrier. U.S Patent 2012/0122219A1 discloses that preparing a porous scaffold by using polyglutamic acid and chondroitin as raw material, and polyglutamic acid and chondroitin have excellent biocompatibility, the porous scaffold prepared can provide cell growth with a three-dimensional microenvironment, and the cells have a strong adhesion and growth ability on the scaffold, showing potential application of the scaffold in tissue engineering. WO 2007/075016A1 discloses preparing hydrogel by using polyglutamic acid and vitamin C as raw material, these two raw material reacts to form a gum under by activation of EDC and NHS, obtaining a solid after freeze drying. The study indicated that this product can significantly inhibit activity of collagenase, and have functions of antioxidation and preventing skin wrinkle, it has extensive application prospect in cosmetic and medical fields. WO2009/157595 A1 discloses that using polyglutamic acid (molecular weight 13000 kDa) obtained by fermentation of Bacillus subtilis as raw material, being cross-linked under the action of γ-ray to prepare a hydrogel, because of cross linking by using γ-ray, there is no any chemical residue, thus the safety of this hydrogel can be ensured, its water absorption and moisture retention ability is very high, it has extensive application prospect in top grade cosmetic field.

Chinese invention patents CN 1629220A and CN 101891954A disclose a method for preparing a hydrogel by using polyglutamic acid as a main raw material, both of these two methods use a cross-linking agent of glycidyl ethers, while water absorption of the gel obtained is high, because of non-biological resource of its cross-linking agent, its biocompatibility is poor, thus it is generally used in farmland water retention and environmental protection and flocculation etc, its application in medical field is greatly restrained. Chinese Patent CN 102585303A discloses a method for preparing a hydrogel by using polylysine as raw material, the chitosan/polylysine hydrogel prepared by this invention simulates the composition and structure of polysaccharide/polypeptide in the nature extracellular matrix, and this hydrogel is expected to be used as a tissue binder as injectable material, but in the preparation process, it is required to conduct chemical modification to chitosan and polylysine respectively, thus the synthesis steps is complicated.

In conclusion, for respective disclosures of preparation of hydrogel by using polyglutamic acid and polylysine as raw material, there are shortcomings such as poor biocompatibility of the cross-linking agent and complicated preparation steps.

### SUMMARY OF THE INVENTION

One technical problem to be resolved by the present invention is to provide a hydrogel based on γ-polyglutamic acid and ε-polylysine cross-linked polymer with a high water absorption, fast swelling rate and good biocompatibility.

Another technical problem to be resolved by the present invention is to provide a preparation method of above-described hydrogel, for this method, the reaction condition is mild, the step is simple and it is not required to conduct further chemical modification to the raw material.

The last technical problem to be resolved by the present invention is to provide a medical application of the above-described hydrogel.

To resolve the above-described technical problems, the technical solution adopted by the present invention is as follows:

A hydrogel based on a γ-polyglutamic acid and ε-polylysine cross-linked polymer, it is obtained by cross-linking of γ-polyglutamic acid with ε-polylysine, and it has a polymer with the following constitutional unit:

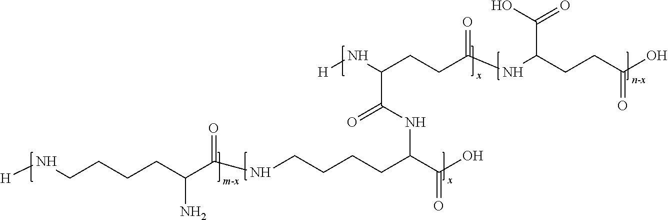

wherein, m is a natural number of 15 to 45, n is a natural number of 3900 to 17000, and x is a natural number of 5 to 40.
wherein, the γ-polyglutamic acid and ε-polylysine are respectively obtained by microbial fermentation.

For instance, γ-polyglutamic acid can be obtained by fermentation using Bacillus subtilis, Bacillus licheniformis, Bacillus megaterium, Bacillus anthracia, Bacillus halodurans etc. Ashiuchi M. Occurrence and biosynthetic mechanism of poly-gamma-glutamic acid. In: Hamano Y editor. Amino-Acid Homopolymers Occurring in Nature [C.] Berlin: Springer; 2010. Preferably, γ-polyglutamic acid is obtained by fermentation using Bacillus subtilis NX-2 (Preserved in China General Microbiological Culture Collection (CGMCC), Preservation Number: CGMCC NO. 0833), specific preparation method can refer to Xu, H., Jiang, M., Li, H., Lu, D. Q., Ouyang, P. K., Efficient production of poly(γ-glutamic acid) by newly isolated Bacillus subtilis NX-2. Process Biochem. 2005. (40), 519-523. or Liang Jinfeng, Xu Hong, Yao Jun, Wu Qun, Pretreatment and isolation and purification process for abstracted fermentation liquor of γ-polyglutamic acid, Food and Fermentation Industry, 2009 (3), 10-15.

For instance, ε-polylysine can be obtained by fermentation using Streptomyces albulus, Streptomyces albidoflavus, Streptomyces rimosus, Streptomyces cyaneus, Kitasatospora sp etc. Nishikawa M, Ogawa K. Distribution of microbes producing antimicrobial ε-poly-L-lysine polymers in soil microflora determined by a novel method. Appl Environ Microbiol., 2002(68): 3575-3581, preferably obtained by using Streptomyces albulus PD-1 (Preserved in China Center for Type Culture Collection (CCTCC), Preservation Number: CCTCC M2011043), the specific preparation method can refer to Hirohara H, Takehara M, Saimura M, Ikezaki A, Miyamoto M (2006). Biosynthesis of poly(ε-L-lysine)s in two newly isolated strains of Streptomyces sp. Appl Microbiol Biotechnol 73: 321-331 or Zhou Jun, Xu Hong, Wang Jun, Yao Zhong, Wang Hui, Ouyang Pingkai, Isolation and purification and structure characterization of the ε-polylysine produced by Kitasatospora PL6-3, Journal of Chemical Engineering 2006 (08): 229-233 or Chen Xiong, Yuan Jinfeng, Wang Shiyu, Zhang Ying, Wang Jinhua. Study on poly-ε-lysine abstracted from fermentation liquor by ion exchange resin, Food Science, 2007 (10): 144-146.

The present invention is not limited in above-described microorganisms and above-described fermentation preparation method. All existing γ-polyglutamic acid and ε-polylysine obtained by using microorganism (including wild strain or gene engineering strain) prior to the filing date of the present patent can be used in the present invention. The polymers formed by cross-linking of γ-polyglutamic acid and ε-polylysine of above-described biology source all have excellent biocompatibility.

Wherein, molecular weight of γ-polyglutamic acid is 500 thousand to 2.2 million Daltons, preferably molecular weight of the γ-polyglutamic acid is 1 million to 1.2 million Daltons; molecular weight of the ε-polylysine is 2000 to 5500 Daltons, preferably molecular weight of the ε-polylysine is 3000 to 4500 Daltons.

A preparation method of hydrogel based on γ-polyglutamic acid and ε-polylysine cross-linked polymer, it includes the following steps:

(1) adding dropwisely a 2-(N-morpholino)ethanesulfonic acid buffer containing ε-polylysine (2-(N-morpholino)ethanesulfonic acid buffer, briefly called MES buffer) into a 2-(N-morpholino)ethanesulfonic acid buffer (2-(N-morpholino)ethanesulfonic acid buffer (briefly called MES buffer) containing γ-polyglutamic acid, stirring and mixing homogeneously;
(2) adding a cross-linking agent into the reaction system obtained in step (1), reacting in an ice bath for 10-120 mins, and reacting for 2-24 hours at room temperature to form a hydrogel;
(3) placing the hydrogel formed in step (2) into a dialysis bag, and dialyzing in de-ionized water until swelling equilibrium, then adopting freeze drying or vacuum drying, to obtain a sponge-like dressing.

In steps (1), the γ-polyglutamic acid and ε-polylysine are respectively obtained by microbial fermentation.

In step (1), molecular weight of γ-polyglutamic acid is 500 thousand to 2.2 million Daltons, preferably molecular weight of γ-polyglutamic acid is 1 million to 1.2 million Daltons; molecular weight of the ε-polylysine is 2000 to 5500 Daltons, preferably molecular weight of the ε-polylysine is 3000 to 4500 Daltons.

In step (1), the MES buffer is a MES buffer of 0.1 mol/L and pH 5.0.

In step (1), a MES buffer containing ε-polylysine is a homogeneous solution, wherein concentration of the ε-polylysine is 20 g/L to 160 g/L, preferably 24 g/L to 60 g/L; a MES buffer containing γ-polyglutamic acid is a homogeneous solution, wherein mass percentage content of the γ-polyglutamic acid is 40 g/L to 200 g/L, preferably 60 g/L to 120 g/L.

In step (2), the cross-linking agent is a combination of 1-(3-dimethylaminopropyl)-3-ethylcarbodiimide (1-ethyl-3-(3-dimethylaminopropyl)carbodiimide hydrochloride, EDC) and N-hydroxysuccinimide (N-hydroxysulfosuccinimide, NHS) (briefly called EDC/NHS), or a combination of 1-(3-dimethylaminopropyl)-3-ethylcarbodiimide (1-ethyl-3-(3-dimethylaminopropyl)carbodiimide hydrochloride, EDC) and N-hydroxysulfosuccinimide (N-hydroxysulfosuccinimide sodium salt, sulfo-NHS) (briefly called EDC/sulfo-NHS), or 1-cyclohexyl-2-morpholinoethylcarbodiimide-p-toluenesulfonate

(1-Cyclohexyl-3-(2-morpholinoethyl)carbodiimidemetho-p-toluenesulfonate, briefly called CMC), or Woodward's Reagent K (i.e. 2-ethyl-5-phenylisoxazolium-3-sulfonate, N-ethyl-3-phenylisoxazolium-3′-sulfonate). When the cross-linking agent is a combination of 1-(3-dimethylaminopropyl)-3-ethylcarbodiimide and N-hydroxysuccinimide, the feeding mole ratio of carboxyl groups included in the γ-polyglutamic acid:amino groups included in the ε-polylysine:1-(3-dimethylaminopropyl)-3-ethylcarbodiimide:N-hydroxysuccinimide is in a range of 1:0.25 to 0.5:0.25 to 1:0.25 to 1, preferably 1:0.4 to 0.5:0.6 to 0.8:0.6 to 0.8. A hydrogel is prepared by using EDC and NHS as activator, EDC can catalyze the raw material to rapidly form amide bond, NHS can increase reaction efficiency and reduce production of rearrangement byproduct, and the activator can be removed by washing the dialysis solution with water.

When the cross-linking agent is a combination of 1-(3-dimethylaminopropyl)-3-ethylcarbodiimide and N-hydroxysulfosuccinimide, and feeding mole ratio of carboxyl groups included in the γ-polyglutamic acid:amino groups included in the ε-polylysine:1-(3-dimethylaminopropyl)-3-ethylcarbodiimide:N-hydroxysulfosuccinimide is in a range of 1:0.25 to 0.5:0.25 to 1:0.25 to 1, preferably 1:0.4 to 0.5:0.5 to 0.8:0.5 to 0.8. When the cross-linking agent is 1-cyclohexyl-2-morpholinoethylcarbodiimide-p-toluenesulfonate, the feeding mole ratio of carboxyl groups included in the γ-polyglutamic acid:amino groups included in the ε-polylysine:1-cyclohexyl-2-morpholinoethylcarbodiimide-p-toluenesulfonate is in a range of 1:0.25 to 0.5:0.25 to 1, preferably 1:0.4 to 0.5:0.3 to 0.6. When the cross-linking agent is Woodward's Reagent K, the feeding mole ratio of carboxyl groups included in the γ-polyglutamic acid:amino groups included in the ε-polylysine:Woodward's Reagent K is in a range of 1:0.25 to 0.5:0.25 to 1, preferably 1:0.4 to 0.5:0.6 to 0.9.

In step (2), reaction time in the ice bath is preferably 30 minutes, and reaction time at room temperature is preferably 2 hours.

In steps (3), temperature of the freeze drying is −40° C.; the temperature of the vacuum drying is 60° C., after the above-described drying process, water content of the product is controlled at 1 to 3 wt %.

The method of the present invention also can conduct a further processing to the sponge-like dressing obtained.

For example, the sponge-like dressing can be grinded and crushed, and split charged with an aluminum composite film, to prepare a xerogel powder.

Or, 1 to 10 fold weight of water is added to the sponge-like dressing to make a soft material, split charged in a polyethylene tube, sealed and packed, to prepare a hydrogel.

Or, 1 to 5 fold weight of water is added into the sponge-like dressing to make a soft material, pressed into a film-coated tablet and placed onto a polyethylene film, drid with a airflow of 70 to 90° C., making its water content at 20 to 60 wt %, and a polyethylene breathable film is laminated, and sealed with an aluminum composite film after cutting, to prepare a gel film.

The hydrogel prepared according to all above-described preparation methods are all within the protection scope of the present invention.

Applications of the hydrogel prepared according to all above-described preparation methods in medical wound dressing are all within the protection scope of the present invention.

Reaction principle diagram of the present invention is seen in FIG. 1.

Compared to the prior art, the significant advantages of the present invention are: using γ-polyglutamic acid and ε-polylysine obtained by microorganism fermentation as the raw material, in vivo degradability and biocompatibility of the raw material are both good, they will not induce rejection of human body, and cell adhesion is good. Reaction condition of the preparation method is mild, step is simple, the hydrogel can be prepared without further chemical modification to the raw material, and water absorption of the hydrogel obtained is high, and swelling rate is fast, and the hydrogel has a good application prospect in wound dressing field.

### BRIEF DESCRIPTION OF THE DRAWINGS

FIG. 1 is a reaction principle diagram of the present invention.

FIG. 2 is a nuclear magnetic resonance spectroscopy (¹H-NMR) of a hydrogel based on γ-polyglutamic acid and ε-polylysine cross-linked polymer.

FIG. 3 is an infrared spectrum (FTIR) of the hydrogel based on γ-polyglutamic acid and ε-polylysine cross-linked polymer in Example 1. (a) is ε-polylysine, (b) is γ-polyglutamic acid, (c) is ε-polylysine-γ-polyglutamic acid hydrogel.

FIG. 4 is an electron microscope image (SEM) of the hydrogel based on γ-polyglutamic acid and ε-polylysine cross-linked polymer (Example 1).

FIG. 5 is a situation of detecting cytocompatibility by a confocal laser scanning microscope (CLSM), the cells grow on the hydrogel scaffold, the living cells are green, and the dead cells are red. a) growth of fibroblasts on the polyglutamic acid hydrogel scaffold; b) growth of fibroblasts on the γ-polyglutamic acid and ε-polylysine cross-linked polymer hydrogel scaffold.

FIG. 6 is back wound healing experiment of domestic rabbit, a) in a control group the back is covered only with a gauze then bandaged; b) a group treated using polyglutamic acid hydrogel dressing; c) a group treated using a γ-polyglutamic acid and ε-polylysine cross-linked polymer hydrogel dressing.

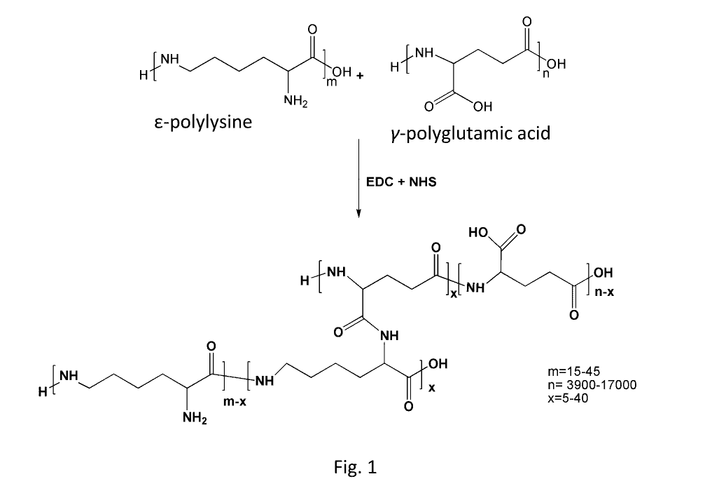

### DESCRIPTION OF THE EMBODIMENTS

According to the following examples, the present invention can be better understood. However, a person skilled in the art will easily understood that, the contents described in the examples are only used to illustrate the present invention, and should not and will not restrain the present invention described in detail in the claims.

The resources of reagents used in the following examples are as follows:

γ-polyglutamic acid and ε-polylysine: purchased from Nanjing Shineking Biological Technology Co., Ltd.;
MES (2-(N-morpholino)ethanesulfonic acid), EDC (1-(3-dimethylaminopropyl)-3-ethylcarbodiimide), NHS (N-hydroxysuccinimide) and Sulfo-NHS (N-hydroxysulfosuccinimide): purchased from Sinopharm Chemical Reagent Co., Ltd;
CMC (1-cyclohexyl-2-morpholinoethylcarbodiimide p-toluenesulfonate) and (2-ethyl-5-phenylisoxazolium-3-sulfonate): purchased from Sigma-Aldrich Company.

The resources of equipments used in the following examples are as follows:

Magnetic stirrer: Type 85-2C, Shanghai Niuhang Instrument and Equipment Co., Ltd.
Freeze dryer: Type FD-1C-50, Beijing Boyikang Experimental Instrument Co., Ltd.
Vacuum drying box: Type YZG-600, Nanjing Yantai Electrical Heating Equipment Co., Ltd.
Infrared spectrometer: Type Nicolet 380, Thermo Company, USA.
NMR spectrometer: Type AVANCE AV-500, Bruker Daltonics Company, USA.

### Example 1

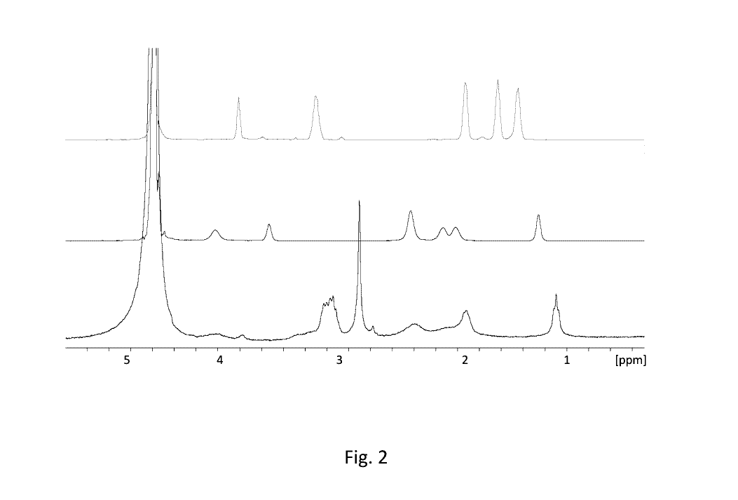

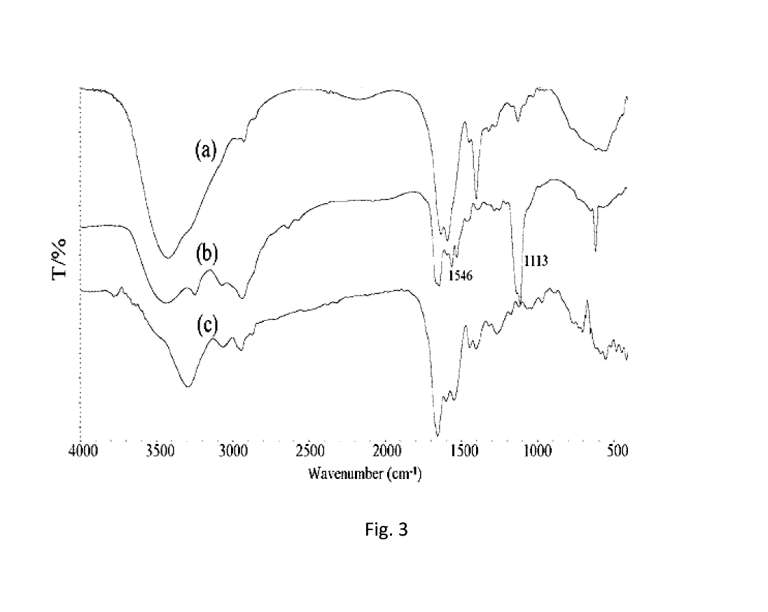

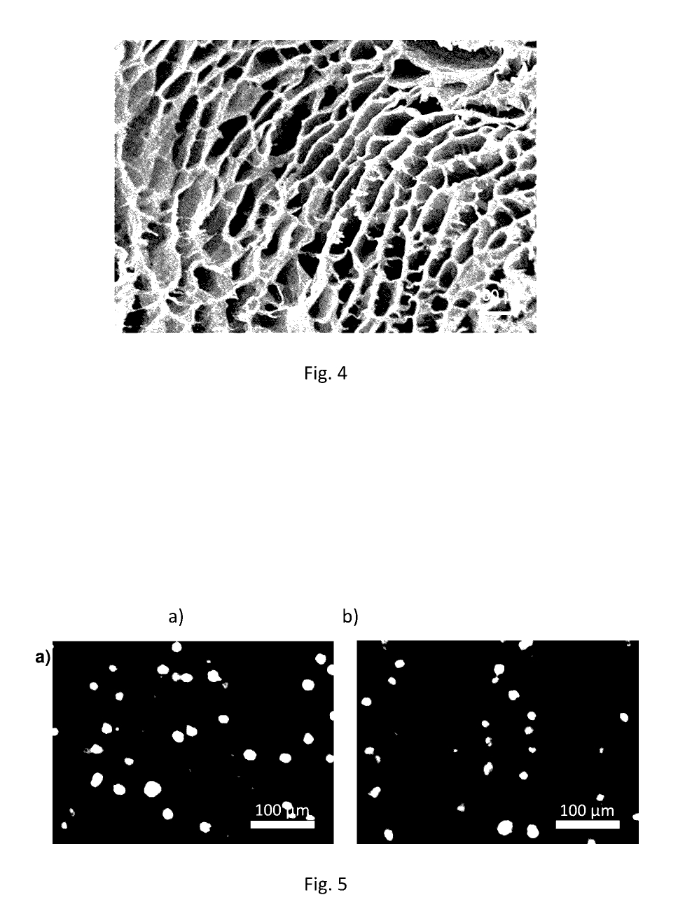

At room temperature 4.0 g γ-polyglutamic acid (1 million to 1.2 million Daltons, including 0.031 mole of carboxyl groups) was dissolved in 50 ml of 0.1 mol/L MES buffer (pH 5.0), and stirred until a clear solution was formed. At room temperature 1.78 g ε-polylysine (3000 to 4500 Daltons, including 0.014 mole of amino groups) was dissolved in 50 ml of 0.1 mol/L MES buffer (pH 5.0), and a ε-polylysine solution was added dropwisely into the polyglutamic acid solution, stirred and making the solution mixed homogenously. 4.17 g (0.0217 mol) EDC and 2.50 g (0.0217 mol) NHS were added into the above-described mixed solution of γ-polyglutamic acid and ε-polylysine, and the feeding mole ratio of carboxyl groups included in the γ-polyglutamic acid:amino group included in the ε-polylysine:EDC:NHS was controlled to 1:0.45:0.7:0.7, and reacted in an ice bath for 30 minutes, then reacted at room temperature for 2 hours to form a hydrogel. The hydrogel formed was placed into a dialysis bag, and dialyzed in deionized water until swelling equilibrium, then freeze drying or vacuum drying was adopted to obtain a sponge-like dressing, swelling rate of the hydrogel formed was 96.6 g/g. Its structure identification is seen in FIG. 2, in the HNMR spectra of γ-polyglutamic acid and ε-polylysine cross-linked polymer hydrogel, it can be seen that chemical binding of γ-polyglutamic acid with ε-polylysine d forms a cross-linked polymer. The infrared spectrum in FIG. 3 also demonstrate forming of γ-polyglutamic acid and ε-polylysine cross-linked polymer hydrogel. Because the ε-polylysine has many free amino groups, characteristic peaks at 1546 cm⁻¹ IA 1113 cm⁻¹ are obvious, after formation of the hydrogel by cross-linking, amino groups of the ε-polylysine reacted with carboxyl groups in the γ-polyglutamic acid to form amide bond, the number of free amino groups during formation of the polymer was greatly reduced, thus the characteristic peaks at these two sites are no longer obvious. Furthermore, one broad peak appearing at site of 3500 to 3300 cm⁻¹ is also a characteristic absorption peak of the hydrogel, it is mainly induced by stretching vibration of the hydroxyl group and N—H stretching vibration on the amide bond. SEM in FIG. 4 shows a surface morphology of the hydrogel prepared, indicating that the hydrogel prepared by the present invention is a three-dimensional porous structure, and the pore size is between 100 and 200 μm, it is suitable to be used as a wound dressing.

### Example 2

At room temperature 5.0 g γ-polyglutamic acid (1 million to 1.2 million Daltons, including 0.039 mol of carboxyl groups) was dissolved in 50 mL of 0.1 mol/L MES buffer (pH=5.0), and stirred until a clear solution was formed. At room temperature 1.99 g ε-polylysine (3000 to 4500 Daltons, including 0.0156 mole of amino groups) was dissolved in 50 mL 0.1 mol/L MES buffer (pH 5.0), a ε-polylysine solution was added dropwisely into the polyglutamic acid solution, stirred and made the solution mixed homogenously. 4.49 g (0.023 mol) EDC and 5.08 g (0.023 mol) sulfo-NHS were added into the above-described mixed solution of γ-polyglutamic acid and ε-polylysine, the feeding mole ratio of carboxyl groups included in the γ-polyglutamic acid:amino groups included in the ε-polylysine:EDC:Sulfo-NHS was controlled to 1:0.4:0.6:0.6, and reacted in an ice bath for 30 minutes, then reacted at room temperature for 2 hours to form a hydrogel. The formed hydrogel was placed in a dialysis bag, and dialyzed in deionized water until swelling equilibrium, then freeze drying or vacuum drying was adopted to obtain a sponge-like dressing, the expansion rate of the hydrogel obtained was 73.8 g/g.

### Example 3

At room temperature 6.0 g γ-polyglutamic acid (1 million to 1.2 million Daltons, including 0.047 mol of carboxyl groups) was dissolved in 50 mL of 0.1 mol/L MES buffer (pH=5.0), and stirred until a clear solution was formed. At room temperature 3.0 g ε-polylysine (3000 to 4500 Daltons, including 0.0235 mol of amino groups) was dissolved in 50 mL 0.1 mol/L MES buffer (pH=5.0), a ε-polylysine solution was added dropwisely into the polyglutamic acid solution, and stirred and made the solution mixed homogenously. 7.95 g (0.0188 mol) CMC was added into the above-described mixed solution of γ-polyglutamic acid and ε-polylysine, and feeding mole ratio of carboxyl groups included in the γ-polyglutamic acid:amino groups included in the ε-polylysine:CMC was controlled to 1:0.5:0.4, and reacted in an ice bath for 30 minutes, then reacted at room temperature for 2 hours to form a hydrogel. The hydrogel formed was placed in a dialysis bag, dialyzed in a deionized water until swelling equilibrium, then freeze drying or vacuum drying was adopted to obtain a sponge-like dressing, the expansion rate of the hydrogel obtained was 48.4 g/g.

### Example 4

At room temperature 4.5 g γ-polyglutamic acid (1 million to 1.2 million Daltons, including 0.035 mol of carboxyl groups) dissolved in a 50 ml of 0.1 mol/L MES buffer (pH 5.0), and stirred to form a clear solution. At room temperature 2.24 g ε-polylysine (3000 to 4500 Daltons, including 0.0175 mol of amino groups) was dissolved in a 50 mL 0.1 mol/L MES buffer (pH 5.0), a ε-polylysine solution was added dropwisely into the polyglutamic acid solution, stirred and made the solution mixed homogenously. 7.08 g (0.028 mol) Woodward's Reagent K was added into the above-described mixed solution of γ-polyglutamic acid and ε-polylysine, and the feeding mole ratio the carboxyl groups included in the γ-polyglutamic acid:amino groups included in the ε-polylysine:Woodward's Reagent K was 1:0.5:0.8, and reacted in an ice bath for 30 minutes, then reacted at room temperature for 2 hours to form a hydrogel. The hydrogel formed was placed into a dialysis bag, and dialyzed in a deionized water until swelling equilibrium, then freeze drying or vacuum drying was adopted to obtain a sponge-like dressing, the expansion rate of the hydrogelo obtained was 52.7 g/g.

### Example 5

The method is the same as Example 1, the difference is controlling the feeding mole ratio of carboxyl groups included in the γ-polyglutamic acid:amino groups included in the ε-polylysine:EDC:NHS to 1:0.25:0.25:0.25, the expansion rate of the hydrogel obtained was 12.5 g/g.

### Example 6

The method is the same as Example 1, the difference is controlling the feeding mole ratio of carboxyl groups included in the γ-polyglutamic acid:amino groups included in the ε-polylysine:EDC:NHS to 1:0.5:1:1, the expansion rate of the hydrogel obtained was 38.6 g/g.

### Example 7

The method is the same as Example 2, the difference is controlling the feeding mole ratio of carboxyl groups in the γ-polyglutamic acid:amino groups included in the ε-polylysine:EDC:Sulfo-NHS to 1:0.25:0.25:0.25, the expansion rate of the hydrogel obtained was 30.2 g/g.

### Example 8

The method is the same as Example 2, the difference is controlling the feeding mole ratio of carboxyl groups included in the γ-polyglutamic acid:amino groups included in the ε-polylysine:EDC:Sulfo-NHS to 1:0.5:1:1, the expansion rate of the hydrogel obtained was 42.3 g/g.

### Example 9

The method is the same as Example 3, the difference is controlling the feeding mole ratio of carboxyl groups in the γ-polyglutamic acid:amino groups included in the ε-polylysine:CMC to 1:0.25:0.25, the expansion rate of the hydrogel obtained was 33.7 g/g.

### Example 10

The method is the same as Example 3, the difference is controlling the feeding mole ratio of carboxyl groups included in the γ-polyglutamic acid:amino groups included in the ε-polylysine:CMC to 1:0.5:1, the expansion rate of the hydrogel obtained was 39.4 g/g.

### Example 11

The method is the same as Example 4, the difference is controlling the feeding mole ratio of carboxyl groups included in the γ-polyglutamic acid:amino groups included in the ε-polylysine:Woodward's Reagent K to 1:0.25:0.25, the expansion rate of the hydrogel obtained was 36.6 g/g.

### Example 12

The method is the same as Example 4, the difference is controlling the feeding mole ratio of carboxyl groups included in the γ-polyglutamic acid:amino groups included in the ε-polylysine:Woodward's Reagent K to 1:0.5:1, the expansion rate of the hydrogel obtained was 43.4 g/g.

### Example 13

The method is the same as Example 4, the difference is reacting in an ice bath for 10 minutes, and reacted at room temperature for 5 hours to form a hydrogel, the expansion rate of the hydrogel obtained was 35.8 g/g.

### Example 14

The method is the same as Example 4, the difference is reacting in an ice bath for 120 minutes, and reacted at room temperature for 24 hours to form a hydrogel, the expansion rate of the hydrogel obtained was 26.9 g/g.

### Comparative Example 1

At room temperature 4.0 g γ-polyglutamic acid (1 million to 1.2 million Daltons, including 0.031 mole of carboxyl groups) were dissolved in 50 mL of 0.1 mol/L MES buffer (pH=5.0), and stirred until a clear solution was formed. 4.17 g (0.0217 mol) EDC and 2.50 g (0.0217 mol) NHS were added into the γ-polyglutamic acid solution, and the feeding mole ratio of carboxyl groups included in the γ-polyglutamic acid:EDC:NHS was controlled to 1:0.7:0.7, and reacted in an ice bath for 30 minutes, then reacted at room temperature for 6 hours to form a hydrogel. The formed hydrogel was placed into a dialysis bag, and dialyzed in deionized water until swelling equilibrium, then freeze drying or vacuum drying was adoped to obtain a sponge-like dressing, the expansion rate of the hydrogel obtained was 3.4 g/g.

### Comparative Example 2

At room temperature, 1.78 g ε-polylysine (3000 to 4500 Daltons, including 0.014 mol of amino groups) was dissolved in 50 mL of 0.1 mol/L MES buffer (pH 5.0), stirred until a clear solution was formed. 4.16 g EDC (0.0217 mmol) and 2.50 g NHS (0.0217 mmol) were added into the ε-polylysine solution, the feeding mole ratio of amino groups included in the ε-polylysine:EDC:NHS was controlled to 0.45:0.7:0.7. Reacted in an ice bath for 30 minutes, then reacted at room temperature for 9 hours to form a hydrogel. The hydrogel formed was placed into a dialysis bag, and dialyzed in a deionized water until swelling equilibrium, then freeze drying or vacuum drying was adopted to obtain a sponge-like dressing, the expansion rate of the hydrogel obtained was 2.8 g/g.

### Comparative Example 3

At room temperature 4.0 g γ-polyglutamic acid (1 milliontol 2 million Daltons, including 0.031 mol of carboxyl groups) was dissolved in 50 mL of 0.1 mol/L MES buffer (pH 5.0), and stirred until a clear solution was formed. At room temperature 1.78 g ε-polylysine (3000 to 4500 Daltons, including 0.014 mol of amino groups) was dissolved in 50 mL 0.1 mol/L MES buffer (pH 5.0), a ε-polylysine solution was added dropwisely into the polyglutamic acid solution, the feeding mole ratio of carboxyl groups included in the γ-polyglutamic acid:amino groups included in the ε-polylysine was controlled to 1:0.45, stirred and making the solution mixed homogenously, but the hydrogel cannot be formed.

### Example 15

The sponge-like dressing in Example 1 to 4 was grinded and crushed, and split charged with an aluminum composite film, to prepare a xerogel powder.

### Example 16

5 g of the sponge-like dressing in Example 1 to 4 was weighed, and 30 g water was added to make a soft material, and split charged in a polyethylene tube, sealed to prepare a hydrogel.

### Example 17

5 g of the sponge-like dressing in Example 1 to 4 was weighed, 20 g of water was added to make a soft material, pressed into a film-coated tablet and placed onto a polyethylene film, and dried by a airflow of 80° C., making its water content being 40 wt %, and a polyethylene breathable film was laminated, sealed with an aluminum composite film after cutting, to prepare a gel film.

### Experiment Example 18

### Cytocompatibility Experiment

3 fold weight of dressing of water was added to the sponge-like dressing in Example 1 to make a soft material hydrogel, and fibroblast was inoculate onto the surface of hydrogel at a concentration of 5×10⁴/cm², and cultured at 37° C. for 6 hours. The cells were stained by a LIVE/DEAD fluorescent reagent, the living cells were stained with green fluorescent material (calcein-AM), and the dead cells were stained with red fluorescent material (EthD-I). Then, the cell survival was observed by a confocal laser scanning microscope (CLSM). See FIG. 5, red fluorescent material in a) are obviously higher than in b), indicating that a considerable amount of cells among the cells on the γ-polyglutamic acid hydrogels caffold are dead, while most of the cells on the γ-polyglutamic acid and ε-polylysine cross-linked polymer hydrogel scaffold are living, this indicated a good biocompatibility of the hydrogel of the present invention.

### Experiment Example 19

### Wound Healing Experiment

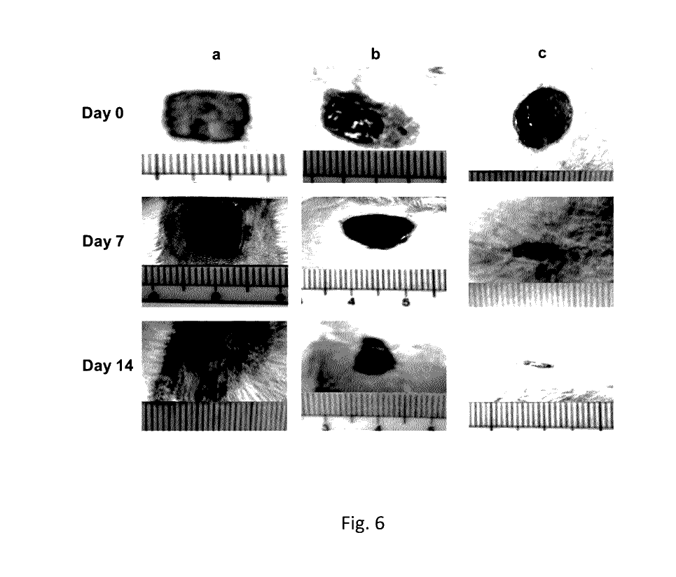

After the back of domestic rabbit was sheared, and unhaired with a sodium sulfide solution for 48 hours, each domestic rabbit was subcutaneously injected 0.5 mL of 0.5% lidocaine injection for local anesthesia, totally at 3 sites, at the local anesthesia site circular skin incisions of about 1.0 cm diameter were sheared at three sites with surgical scissors, disinfected with a 70 v/v % ethanol, The wound dressing in Example 1 was coated at the wound, and covered with a wax degreased cotton gauze, and bandaged, in the control groups one group was covered only with a gauze then bandaged, the another group was coated with a γ-polyglutamic acid hydrogel dressing. During experiment each group had no bacteria infection, and the wound healing condition were observed respectively after 0, 7, 14 days. See FIG. 6, with extension of treatment time, in all three groups the wound of the domestic rabbits were all healed, the hydrogel dressing treated groups were all superior to the group treated only with the gauze; in the group treated by using γ-polyglutamic acid and ε-polylysine cross-linked polymer hydrogel dressing, the wound area was only 40% of that of control group of γ-polyglutamic acid hydrogel, the wound was obviously decreased, and the surface was smooth and flat, showing a good biocompatibility and ability of promoting wound healing.

Comprehensive evaluation: the γ-polyglutamic acid/ε-polylysine hydrogel wound dressing of the present invention has a good biocompatibility, and contributes to cell adhesion and growth, it has a promotion effect to wound healing, and can effectively reduce leakage of tissue fluid, having an extensive application prospect in the medical wound dressing field.

What is claimed is:

1. A hydrogel based on a cross-linked polymer of γ-polyglutamic acid and ε-polylysine, characterized in that, it is obtained by cross-linking of the γ-polyglutamic acid with the ε-polylysine, and it is a polymer having the following constitutional unit: 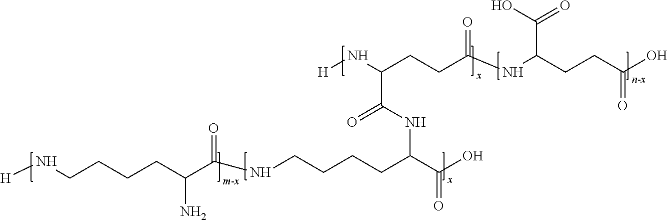
wherein, m is a natural number of 15 to 45, n is a natural number of 3900 to 17000, and x is a natural number of 5 to 40.

2. The hydrogel according to claim 1, characterized in that, the γ-polyglutamic acid and ε-polylysine are obtained by microbial fermentation, respectively.

3. The hydrogel according to claim 1 or 2, characterized in that, molecular weight of the γ-polyglutamic acid is 500 thousand to 2.2 million Daltons, molecular weight of the ε-polylysine is 2000 to 5500 Daltons.

4. A process for preparing a hydrogel with a γ-polyglutamic acid and a ε-polylysine, characterized in that, it comprises the following steps:
(1) adding dropwisely a 2-(N-morpholino)ethanesulfonic acid buffer containing the ε-polylysine into a 2-(N-morpholino)ethanesulfonic acid buffer containing the γ-polyglutamic acid, and stirring and mixing homogeneously;
(2) adding a cross-linking agent into the mixture obtained in step (1), reacting in an ice bath for 10 to 120 min, then reacting for 2 to 24 hours at room temperature to form said hydrogel;
(3) placing the hydrogel formed in step (2) into a dialysis bag, and dialyzing in deionized water until swelling equilibrium, then adopting freeze drying or vacuum drying, to obtain a sponge-like dressing.

5. The process according to claim 4, characterized in that, in step (1), the γ-polyglutamic acid and ε-polylysine are obtained by microbial fermentation, respectively.

6. The process according to claim 4, characterized in that, in step (1), molecular weight of the γ-polyglutamic acid is 500 thousand to 2.2 million Daltons, molecular weight of the ε-polylysine is 2000 to 5500 Daltons.

7. The process according to claim 4, characterized in that, in step (1), the MES buffer is of 0.1 mol/L and pH 5.0.

8. The process according to claim 4, characterized in that, in step (1), the MES buffer containing the ε-polylysine is a homogeneous solution, wherein concentration of the ε-polylysine is 20 g/L to 160 g/L; the MES buffer containing γ-polyglutamic acid is a homogeneous solution, wherein mass percentage content of the γ-polyglutamic acid is 40 g/L to 200 g/L.

9. The process according to claim 4, characterized in that, in step (2), the cross-linking agent is selected from a group consisting of a combination of 1-(3-dimethylaminopropyl)-3-ethylcarbodiimide and N-hydroxysuccinimide, a combination of 1-(3-dimethylaminopropyl)-3-ethylcarbodiimide and N-hydroxysulfosuccinimide, 1-cyclohexyl-2-morpholinoethylcarbodiimide-p-toluenesulfonate and Woodward's Reagent K.

10. The process according to claim 9, characterized in that, in steps (2), the cross-linking agent is a combination of 1-(3-dimethylaminopropyl)-3-ethylcarbodiimide and N-hydroxysuccinimide, wherein the mole ratio of carboxyl groups included in the γ-polyglutamic acid:amino groups included in the ε-polylysine:1-(3-dimethylaminopropyl)-3-ethylcarbodiimide:N-hydroxysuccinimide is 1:0.25 to 0.5:0.25 to 1:0.25 to 1.

11. The process according to claim 9, characterized in that, in step (2), the cross-linking agent is a combination of 1-(3-dimethylaminopropyl)-3-ethylcarbodiimide and N-hydroxysulfosuccinimide, wherein the mole ratio of carboxyl groups included in the γ-polyglutamic acid:amino groups included in the ε-polylysine:1-(3-dimethylaminopropyl)-3-ethylcarbodiimide:N-hydroxysulfosuccinimide is 1:0.25 to 0.5:0.25 to 1:0.25 to 1.

12. The process according to claim 9, characterized in that, in step (2), the cross-linking agent is 1-cyclohexyl-2-morpholinoethylcarbodiimide-p-toluenesulfonate, wherein the mole ratio of carboxyl groups included in the γ-polyglutamic acid:amino groups included in the ε-polylysine:1-cyclohexyl-2-morpholinoethylcarbodiimide-p-toluenesulfonatebeis is 1:0.25 to 0.5:0.25 to 1.

13. The process according to claim 9, characterized in that, in step (2), the cross-linking agent is Woodward's Reagent K, wherein the mole ratio of carboxyl groups included in the γ-polyglutamic acid:amino groups included in the ε-polylysine:Woodward's Reagent K is 1:0.25 to 0.5:0.25 to 1.

14. The process according to claim 4, characterized in that, grinding and crushing the sponge-like dressing, and split charging with an aluminum composite film, a xerogel powder is yielded.

15. The process according to claim 4, characterized in that, adding 1 to 10 fold weight of water to the sponge-like dressing to make a soft material, split charging in a polyethylene tube, sealing and packing, the hydrogel is yielded.

16. The process according to claim 4, characterized in that, adding 1 to 5 fold weight of water to the sponge-like dressing to prepare a soft material, pressing into a film-coated tablet and placing onto a polyethylene film, drying by an airflow of 70 to 90.0 making its water content being 20 to 60 wt %, and laminating a polyethylene breathable film, after cutting sealing with an aluminum composite film, thereby a gel film is made.

17. A hydrogel is prepared by the process of claim 4.

18. A process for preparing a medical wound dressing uses the hydrogel of claim 1 or claim 17.
